(19) United States
(12) Patent Application Publication
(10) Pub. No.: US 2018/0310914 A1
(43) Pub. Date: Nov. 1, 2018

(54) 3D Ultrasound Imaging System for Nerve Block Applications

(71) Applicant: Avent, Inc., Alpharetta, GA (US)

(72) Inventors: Kenneth C. Hsu, Tustin, CA (US); Justin J. Coker, Laguna Niguel, CA (US)

(21) Appl. No.: 15/765,849

(22) PCT Filed: Sep. 15, 2016
(86) PCT No.: PCT/US2016/051890

§ 371 Date: Apr. 4, 2018

Related U.S. Application Data
(60) Provisional application No. 62/247,917, filed on Oct. 29, 2015.

(51) Int. Cl. A61B 8/00; A61B 8/14; A61B 8/08; G10K 11/35

(52) U.S. Cl. CPC A61B 8/4461; A61B 8/54; A61B 8/466; G10K 11/355; A61B 8/4494; A61B 8/5246; A61B 8/145

(57) ABSTRACT

The present disclosure is directed to an ultrasound imaging system for generating 3D images. The system includes an ultrasound probe having a transducer housing and a transducer transmitter. The housing has a body extending from a proximal end to a distal end along a longitudinal axis. The distal end includes an internal cavity that extends, at least, from a first side to a second side along a lateral axis of the housing. The transmitter is mounted to the first and second sides within the cavity and is configured to rotate about the lateral axis for scanning of an ultrasound beam. Thus, during operation, the transmitter is free to rotate in a clockwise direction and/or a counter-clockwise direction about the lateral axis so as to continuously scan two-dimensional (2D) images. The system may also include a controller configured to receive and organize the 2D images in real-time and generate a 3D image based on the 2D images.

### RELATED APPLICATIONS

This application claims the benefit of U.S. Provisional Applications No. 62/247,917, filed Oct. 29, 2015, which is incorporated by reference herein in its entirety.

### FIELD OF THE INVENTION

The present invention relates in general to 3D ultrasound imaging systems, and more particularly to a 3D medical ultrasound imaging system for nerve block applications,

### BACKGROUND OF THE INVENTION

In conventional ultrasound imaging, a focused beam of ultrasound energy is transmitted into body tissues to be examined and the returned echoes are detected and plotted to form an image. More specifically, some modern ultrasound systems have three-dimensional (3D) capabilities that scan a pulsed ultrasound beam in two side-wards directions relative to a beam axis. Time of flight conversion gives the image resolution along the beam direction (range), while image resolution transverse to the beam direction is obtained by the side-wards scanning of the focused beam. With such 3D imaging, a user can collect volume ultrasound data from an object and visualize any cross-section of the object through computer processing. This enables selection of the best two-dimensional (2D) image planes for a diagnosis. Even still, such 3D systems are still limited to a 2D view.

Such systems can be problematic for nerve blocks and/or various other medical procedures, since it is often desirable to locate anatomical structures and devices in a 3D space. Still additional 3D systems for addressing such limitations may include arrayed transducers, which include many ultrasound transmitters and receivers. Such transducers, however, can be expensive and bulky.

Thus, the art is continuously seeking new and improved 3D ultrasound probes. More specifically, a low cost, simplified 3D ultrasound probe that enhances the effectiveness of nerve block procedures by allowing anesthesiologists to better locate structures and/or devices would be advantageous. In addition, a 3D ultrasound probe that maintains the current transducer profile, rather than a bulky arrayed transducer, would be welcomed in the art.

### BRIEF SUMMARY OF THE INVENTION

Aspects and advantages of the invention will be set forth in part in the following description, or may be obvious from the description, or may be learned through practice of the invention.

In one aspect, the present disclosure is directed to an ultrasound imaging system. The ultrasound imaging system includes an ultrasound probe having a transducer housing and a transducer transmitter. The transducer housing has a body extending from a proximal end to a distal end along a longitudinal axis. The distal end includes an internal cavity that extends, at least, from a first side to a second side along a lateral axis of the transducer housing. Thus, the transducer transmitter is mounted to the first and second sides within the cavity of the housing. Further, the transducer transmitter is configured to rotate about the lateral axis for scanning of an ultrasound beam. Thus, during operation, the transducer transmitter is free to rotate in a clockwise direction and/or a counter-clockwise direction about the lateral axis so as to continuously scan two-dimensional (2D) images. The ultrasound imaging system may also include a controller configured to receive and organize the 2D images in real-time and generate a three-dimensional (3D) image based on the 2D images.

In one embodiment, the ultrasound imaging system may also include a user interface configured to display the 3D image. More specifically, in certain embodiments, the user interface is configured to allow a user to manipulate the 3D image according to one or more user preferences.

In another embodiment, the transducer transmitter is configured to emit (or send) and/or receive ultrasound beams. More specifically, in certain embodiments, the transducer transmitter may have a gimbal configuration. For example, in particular embodiments, the transducer transmitter may include at least one plate mounted to a shaft that is rotatable about the lateral axis. Further, the shaft may include a first end and a second end, with the first end being mounted to the first side of the internal cavity and the second end being mounted to the second side. Moreover, in particular embodiments, the plate may be constructed of a piezoelectric material. In additional embodiments, the plate may have any suitable shape, including but not limited to a substantially rectangular shape or a square shape.

In further embodiments, the transducer transmitter may be rotatable by a motor configured within the body of the transducer housing.

In yet another embodiment, the distal end of the body of the transducer housing may include a lens having a linear configuration, wherein the transducer transmitter is configured adjacent to the lens.

In additional embodiments, the cavity of the distal end of the body of the transducer housing may extend through the proximal end of the body. In further embodiments, the distal end of the body of the transducer housing may be wider than the proximal end or vice versa. In still additional embodiments, the proximal and distal ends of the body of the housing may have substantially the same width.

In another aspect, the present disclosure is directed to an ultrasound probe for imaging. The probe includes a transducer housing with a transducer transmitter configured therein. The transducer housing includes a body extending from a proximal end to a distal end along a longitudinal axis. The distal end includes an internal cavity that extends, at least, from a first side to a second side along a lateral axis of the transducer housing. The transducer transmitter is mounted to the first and second sides within the cavity. Further, the transducer transmitter is configured to rotate about the lateral axis for scanning of an ultrasound beam. Thus, during operation, the transducer transmitter is free to rotate in a clockwise direction and/or a counter-clockwise direction about the lateral axis so as to continuously scan two-dimensional (2D) images that can be used to generate a three-dimensional (3D) image. It should be understood that the ultrasound probe may be further configured with any of the additional features as described herein.

In another aspect, the present disclosure is directed to a method of generating a three-dimensional (3D) ultrasound image. The method includes aligning an ultrasound probe at a target site of a patient. As mentioned, the ultrasound probe includes a transducer housing with a transducer transmitter mounted therein. Further, the transducer transmitter is configured to rotate about a lateral axis of the housing. The method also includes continuously scanning, via the transducer transmitter, two-dimensional (2D) images of the target site by rotating the transducer transmitter about the lateral axis in a clockwise direction and/or a counter-clockwise direction. Further, the method includes receiving and organizing, via a controller, the 2D images in real-time. The method also includes generating, via the controller, a three-dimensional (3D) image based on the 2D images.

In one embodiment, the method may also include displaying, via a user interface, the 3D image to a user. More specifically, in certain embodiments, the method may include allowing, via the user interface, a user to manipulate the 3D image according to one or more user preferences.

In additional embodiments, the transducer transmitter may include at least one plate mounted to a shaft that is rotatable about the lateral axis. Thus, in certain embodiments, the method may include mounting the shaft within a cavity of the transducer housing such that the shaft is substantially parallel to the lateral axis. In particular embodiments, the method may include constructing the plate from a piezoelectric material.

In further embodiments, the method may include rotating the transducer transmitter by a motor configured within the transducer housing.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a schematic diagram of one embodiment of an ultrasound imaging system according to the present disclosure;

FIG. 2 illustrates a block diagram of one embodiment of suitable components that may be included in a controller of an ultrasound imaging system according to the present disclosure;

FIG. 3 illustrates a front view of one embodiment of an ultrasound probe of an ultrasound imaging system according to the present disclosure;

FIG. 4 illustrates a side view of the ultrasound probe of FIG. 3;

FIG. 5 illustrates a detailed, internal view of the distal end of the ultrasound probe of FIG. 3;

FIG. 6 illustrates another detailed, internal view of the distal end of the ultrasound probe of FIG. 3, particularly illustrating an ultrasound beam being generated by the probe for a nerve block procedure;

FIG. 7 illustrates an internal, front view of the distal end of the ultrasound probe of FIG. 5;

FIG. 8 illustrates another side view of the ultrasound probe of FIG. 3, particularly illustrating an ultrasound beam being generated by the probe for a nerve block procedure; and

FIG. 9 illustrates a flow diagram of one embodiment of a method of generating a three-dimensional (3D) ultrasound image according to the present disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

Generally, the present disclosure is directed to an ultrasound imaging system having an improved ultrasound probe. For example, the ultrasound probe has a transducer housing with a transducer transmitter mounted therein. More specifically, the transducer housing has a body extending from a proximal end to a distal end along a longitudinal axis thereof. The distal end includes an internal cavity that extends, at least, from a first side to a second side along a perpendicular, lateral axis of the transducer housing. The transducer transmitter is mounted to the first and second sides within the internal cavity and is configured to rotate about the lateral axis for scanning of an ultrasound beam. Thus, during operation, the transducer transmitter is free to rotate in a clockwise direction and/or a counter-clockwise direction about the lateral axis so as to continuously scan two-dimensional (2D) images. The ultrasound imaging system may also include a controller configured to receive and organize the 2D images, e.g. in real-time, and generate a three-dimensional (3D) image based on the 2D images. Such a system can be particularly advantageous during nerve block applications as the ultrasound probe of the present disclosure can be placed at a target site of a patient (e.g. on an outer surface of the patient's skin where a nerve block procedure is to be performed at a nerve or nerve bundle therebeneath) and can remain in the same location as the probe generates the 3D image.

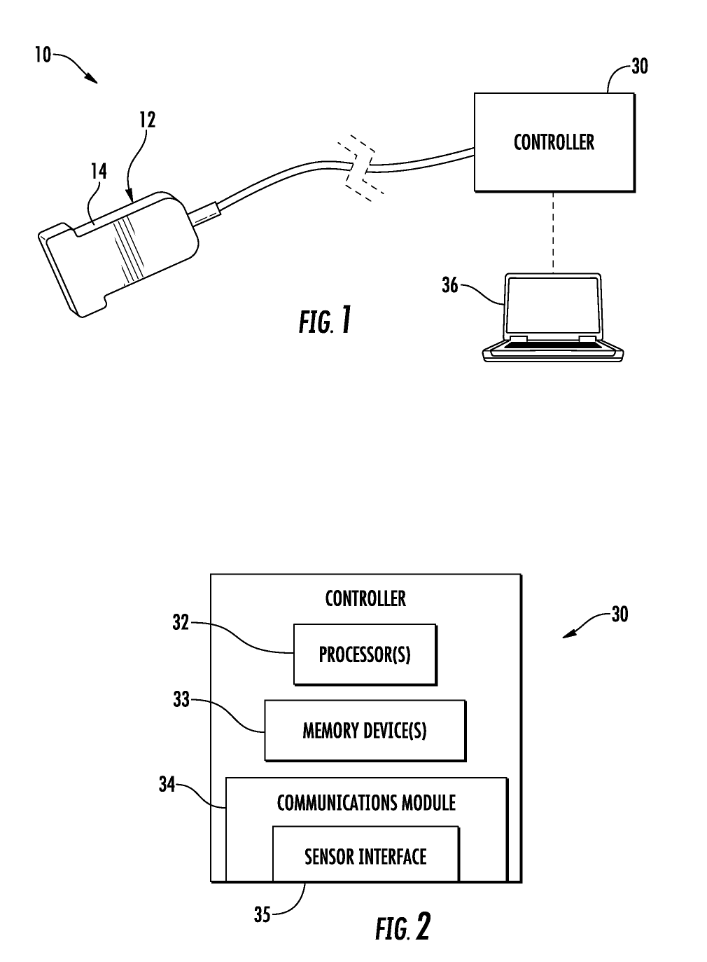

Referring now to the drawings, FIG. 1 illustrates a schematic diagram of one embodiment of an ultrasound imaging system 10 according to the present disclosure. As shown, the ultrasound imaging system 10 includes an ultrasound probe 12. More specifically, as shown in FIGS. 5 and 6, the ultrasound probe 12 has a transducer housing 14 and a transducer transmitter 16 mounted therein. Further, as shown in FIGS. 3-8, the housing 14 generally has a body 15 extending from a proximal end 17 to a distal end 19 along a longitudinal axis 18 thereof. In addition, as shown particularly in FIGS. 3 and 5-6, the distal end 19 includes an internal cavity 20 that extends, at least, from a first side 22 to a second side 24 along a lateral axis 26 of the housing 14. Further, as shown in FIG. 3, the longitudinal axis 18 is generally perpendicular to the lateral axis 26.

In additional embodiments, as shown in FIGS. 5 and 6, the internal cavity 20 at the distal end 19 of the body 15 of the transducer housing 14 may extend through the proximal end 17 of the body 15. In other words, as shown in FIGS. 5-7, the internal cavity 20 may encompass substantially the entire housing 14. In addition, as shown generally in the figures, the distal end 19 of the body 15 of the housing 14 may be wider than the proximal end 17 of the body 15, e.g. such that the proximal end 17 of the body 15 can be easily gripped by a user. Alternatively, the distal end 19 of the body 15 of the housing 14 may be narrower than the proximal end 17 of the body 15. In still another embodiment, the proximal and distal ends 17, 19 of the body 15 of the housing 14 have substantially the same width along the longitudinal axis 18.

In addition, as shown in FIGS. 5 and 6, the distal end 19 of the body 15 of the transducer housing 14 may also include a lens 21 having any suitable configuration. Thus, the lens 21 is configured to allow passage of the ultrasonic beams 42 therethrough. For example, as shown, the lens 21 may have a linear configuration. In further embodiments, the lens 21 may have a convex configuration. Thus, as shown, the transducer transmitter 16 may be configured adjacent to the lens 21.

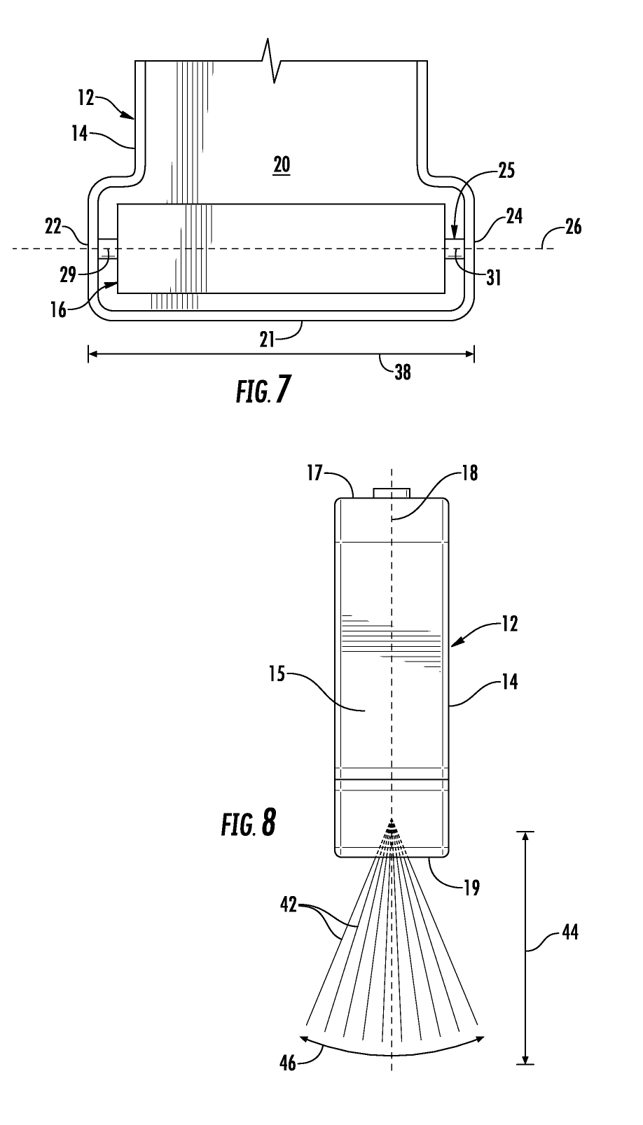

As is generally understood, the transducer transmitter 16 is configured to emit and/or receive ultrasound beams. For example, as shown in FIGS. 5 and 6, the transducer transmitter 16 may be mounted to the first and second sides 22, 24 of the internal cavity 20 such that the transmitter 16 is configured to rotate about the lateral axis 26 for scanning ultrasound beams. More specifically, in certain embodiments, the transducer transmitter 16 may have a gimbal configuration. As used herein, a “gimbal configuration” generally refers to a pivoted support that allows for rotation of an object about a single axis. Thus, as shown in FIGS. 5 and 6, the transducer transmitter 16 may include at least one plate 23 mounted to a shaft 25 that is rotatable about the lateral axis 26. Further, as shown in FIG. 7, the shaft 25 may include a first end 29 and a second end 31. More specifically, as shown, the first end 29 of the shaft 25 may be mounted to the first side 22 of the internal cavity 20 of the transducer housing 14, whereas the second end 31 may be mounted to the opposing, second side 24 of the internal cavity 20. As such, the plate 23 can be mounted along any portion of the length 38 of the shaft 25. For example, as shown, the plate 23 extends substantially the length 38 of the shaft 25. In addition, as shown, the plate 23 may have a solid configuration (as shown) or may have a segmented configuration.

It should be understood that the plate 23 may be constructed of any suitable material configured to scan ultrasound beams. For example, in particular embodiments, the plate 23 may be constructed of a piezoelectric material. In additional embodiments, the plate 23 may have any suitable shape. For example, as shown, the plate 23 has a generally rectangular shape. In another embodiment, the plate 23 may have a square shape.

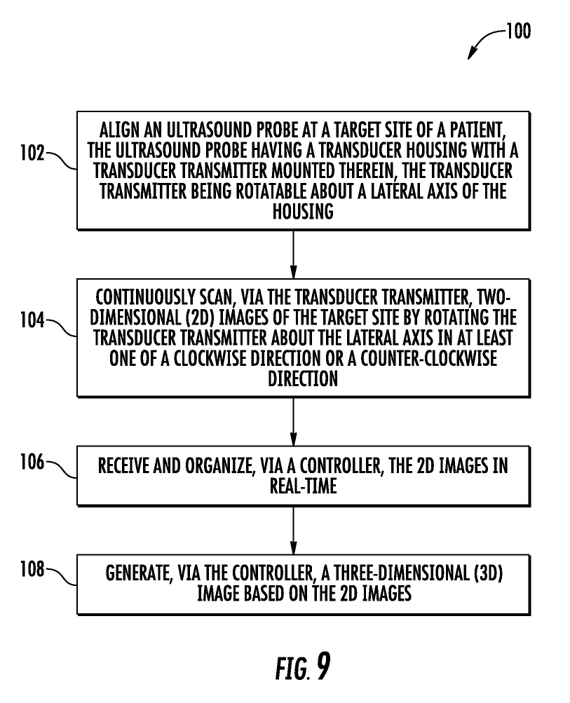

Thus, during operation, the probe 12 can be placed at a target site of the patient and while maintaining the probe 12 in its initial position, the plate 23 of the transducer transmitter 16 is free to rotate about the shaft 25 in a clockwise direction (as indicated by arrow 27 in FIG. 5) and/or a counter-clockwise direction (as indicated by arrow 28 in FIG. 5) about the lateral axis 26 so as to continuously scan two-dimensional (2D) images in an ultrasound plane 40, e.g. by generating multiple ultrasound beams 42 (FIGS. 6 and 8). More specifically, in certain embodiments, the transducer transmitter 16 may be rotated by a motor configured within the body 15 of the transducer housing 14. As such, the probe 12 can be particularly advantageous for nerve block applications as the plate 23 is configured to generate particularly useful images at a predetermined depth 44, which corresponds to a location of a nerve or nerve bundle. Further, as shown in FIG. 8, the width 46 of the image may be adjusted based on a variety of design factors. For example, the width 46 of the image can be modified by changing the dimensions of the plate 23 (e.g. length, width, height, etc.), the speed of rotation of the shaft 25, the angle of the plate 23 with respect to the shaft 25, or similar.

Referring back to FIGS. 1 and 2, the ultrasound imaging system 10 may also include a controller 30 configured to receive and organize the 2D images generated by the transducer transmitter 16 in real-time and generate a three-dimensional (3D) image based on the 2D images. More specifically, as shown in FIG. 2, there is illustrated a block diagram of one embodiment of suitable components that may be included within the controller 30 in accordance with aspects of the present subject matter. As shown, the controller 30 may include one or more processor(s) 32 and associated memory device(s) 33 configured to perform a variety of computer-implemented functions (e.g., performing the methods, steps, and the like and storing relevant data as disclosed herein). Additionally, the controller 30 may also include a communications module 34 to facilitate communications between the controller 30 and the various components of the system 10. Further, the communications module 34 may include a sensor interface 35 (e.g., one or more analog-to-digital converters) to permit signals transmitted from the probe 12 to be converted into signals that can be understood and processed by the processors 32. In addition, as shown, the ultrasound imaging system 10 may also include a user interface 36 (FIG. 1) configured to display the 3D image. More specifically, in certain embodiments, the user interface 36 may be configured to allow a user to manipulate the 3D image according to one or more user preferences.

Referring now to FIG. 9, a flow diagram of one embodiment of a method 100 of generating a three-dimensional (3D) ultrasound image is illustrated. As shown at 102, the method 100 includes aligning an ultrasound probe 12 at a target site of a patient. For example, the probe 12 may be aligned at a location that corresponds to a nerve or nerve bundle where a nerve block procedure is to be performed. As mentioned, the ultrasound probe 12 includes a transducer housing 14 with a transducer transmitter 16 mounted therein. Further, the transducer transmitter 16 is configured to rotate about the lateral axis 26 of the housing 14. Thus, as shown at 104, the method 100 includes continuously scanning, via the transducer transmitter 16, two-dimensional (2D) images (e.g. as indicated by ultrasonic beams 42) of the target site by rotating the transducer transmitter 16 about the lateral axis 26 in a clockwise direction 27 and/or a counter-clockwise direction 28. As shown at 106, the method 100 includes receiving and organizing, via a controller, the 2D images in real-time. As shown at 108, the method 100 includes generating, via the controller, a three-dimensional (3D) image based on the 2D images.

In addition, in one embodiment, the method 100 may also include displaying, via a user interface 36, the 3D image to a user. More specifically, in certain embodiments, the method 100 may include allowing, via the user interface 36, a user to manipulate the 3D image according to one or more user preferences.

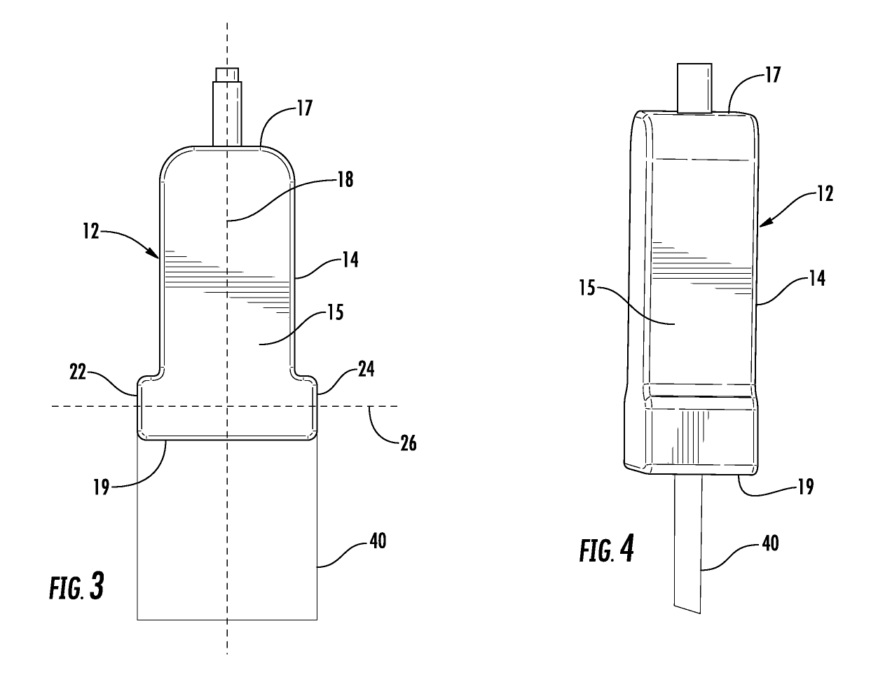

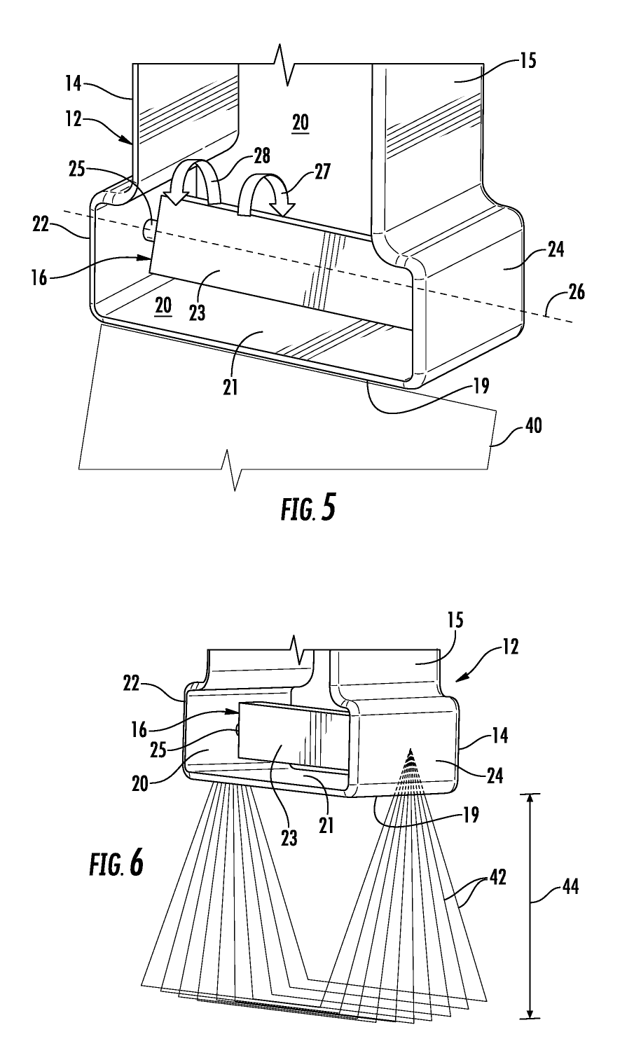

In additional embodiments, as mentioned in reference to FIG. 4, the transducer transmitter 16 may include at least one plate 23 mounted to a shaft 25 that is rotatable about the lateral axis 26. Thus, in certain embodiments, the method 100 may include mounting the shaft 26 within the internal cavity 20 of the transducer housing 14 such that the shaft 25 is substantially parallel to the lateral axis 26. In further embodiments, the method 100 may include rotating the transducer transmitter 16 by a motor configured within the transducer housing 14 (not shown). Accordingly, when the probe 12 is located at a target site of the patient, the transducer transmitter 16 is configured to continuously rotate so as to generate a 3D image of an object at a depth 44. Further, it should be understood that the ultrasound imaging system 10 may include any of the additional features as described herein.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

1. An ultrasound imaging system, comprising:
an ultrasound probe comprising:
a transducer housing comprising a body extending from a proximal end to a distal end along a longitudinal axis, the distal end comprising an internal cavity that extends, at least, from a first side to a second side along a lateral axis of the transducer housing; and
a transducer transmitter mounted to the first and second sides within the cavity, the transducer transmitter being rotatable about the lateral axis for scanning of an ultrasound beam, wherein, during operation, the transducer transmitter is free to rotate in a clockwise direction and a counter-clockwise direction about the lateral axis so as to continuously scan two-dimensional (2D) images; and,
a controller configured to receive and organize the 2D images in real-time and generate a three-dimensional (3D) image based on the 2D images.

2. The ultrasound imaging system of claim 1, further comprising a user interface configured to display the 3D image, the user interface configured to allow a user to manipulate the 3D image according to one or more user preferences.

3. The ultrasound imaging system of claim 1, wherein the transducer transmitter is configured to emit and receive ultrasound beams.

4. The ultrasound imaging system of claim 1, wherein the transducer transmitter comprises a gimbal configuration.

5. The ultrasound imaging system of claim 4, wherein the transducer transmitter comprises at least one plate mounted to a shaft that is rotatable about the lateral axis, the shaft comprising a first end and a second end, the first end being mounted to the first side of the cavity of the housing and the second end being mounted to the second side of the cavity.

6. The ultrasound imaging system of claim 5, wherein the at least one plate is constructed of a piezoelectric material.

7. The ultrasound imaging system of claim 5, wherein the at least one plate of the transducer transmitter comprises a substantially rectangular shape.

8. The ultrasound imaging system of claim 1, wherein the transducer transmitter is rotatable by a motor configured within the body of the transducer housing.

9. The ultrasound imaging system of claim 1, wherein the cavity of the distal end of the body of the transducer housing extends through the proximal end of the body.

10. The ultrasound imaging system of claim 1, wherein the distal end of the body of the transducer housing comprises a lens having a linear configuration, and wherein the transducer transmitter is configured adjacent to the lens.

11. The ultrasound imaging system of claim 1, wherein the distal end of the body of the transducer housing is wider than the proximal end.

12. An ultrasound probe for imaging, comprising:
a transducer housing comprising a body extending from a proximal end to a distal end along a longitudinal axis, the distal end comprising an internal cavity that extends, at least, from a first side to a second side along a lateral axis of the transducer housing; and
a transducer transmitter mounted to the first and second sides within the cavity, the transducer transmitter configured to emit and receive ultrasound beams, the transducer transmitter being rotatable about the lateral axis for scanning of an ultrasound beam, wherein, during operation, the transducer transmitter is free to rotate in a clockwise direction and a counter-clockwise direction about the lateral axis so as to continuously scan two-dimensional (2D) images that can be used to generate a three-dimensional (3D) image.

13. The ultrasound probe of claim 12, wherein the transducer transmitter comprises a gimbal configuration.

14. The ultrasound probe of claim 12, wherein the transducer transmitter comprises at least one plate mounted to a shaft that is rotatable about the lateral axis, the shaft comprising a first end and a second end, the first end being mounted to the first side of the cavity of the housing and the second end being mounted to the second side of the cavity.

15. The ultrasound probe of claim 14, wherein the at least one plate is constructed of a piezoelectric material.

16. The ultrasound probe of claim 14, wherein the at least one plate comprises a substantially rectangular shape.

17. The ultrasound probe of claim 12, wherein the transducer transmitter is rotatable by a motor configured within the body of the transducer housing.

18. A method of generating a three-dimensional ultrasound image, the method comprising:
aligning an ultrasound probe at a target site of a patient, the ultrasound probe having a transducer housing with a transducer transmitter mounted therein, the transducer transmitter comprising at least one plate mounted to a shaft that is substantially parallel to a lateral axis of the housing such that the plate is rotatable about the lateral axis;
continuously scanning, via the transducer transmitter, two-dimensional (2D) images of the target site by rotating the transducer transmitter about the lateral axis in at least one of a clockwise direction or a counter-clockwise direction;
receiving and organizing, via a controller, the 2D images in real-time;
generating, via the controller, a three-dimensional (3D) image based on the 2D images; and
displaying, via a user interface, the 3D image to a user.

19. The method of claim 18, further comprising allowing, via the user interface, a user to manipulate the 3D image according to one or more user preferences.

20. The method of claim 18, further comprising rotating the plate of the transducer transmitter by a motor configured within the transducer housing.
